SEQUENCE LISTING (16 sequences)

SEQ ID NO: 1 - DNA, 490 bp, Gallus domesticus
  CDS (64)..(267)
  misc_feature (258)..(258): n is T or C
  misc_feature (294)..(294): n is T or C
agcttctgaacaccgtcaggcatcttcacagctgcaaaggctattccacagcagaggacaatcatgagaatccttttcttccttgttgctgttctcttcttcctcttccaggctgctccagcttacagccaagaagacgctgacaccttagcatgcaggcagagccacggctcctgctcttttgttgcatgccgtgctccttcagttgacattgggacctgccgtggtgggaagctgaaatgctgcaaatgggcaccnagctcctaaatctgagacctcactgaccacgcagtnaagagccctggaaatctgagatgtctcctgccttatgacatcactggatctttgaactttggtacaaacccaaggagcctttcccaatggggtgaagagtcctgggagcacgagaagatctgaggaggaattcttgtacctctgatacagatttgcagcttcatttctaataaaaacaattcaaagtgaaaccaca

SEQ ID NO: 2 - protein, 67 aa, Gallus domesticus
MRILFFLVAVLFFLFQAAPAYSQEDADTLACRQSHGSCSFVACRAPSVDIGTCRGGKLKCCKWAPSS

SEQ ID NO: 3 - DNA, 3050 bp, Gallus gallus
gtcatttggtccttgttcaggtcaggcatcttcaaatgtgttgggttgcaacatcttcatacatcaccaatctctacagtcatagggcaaagaactctcaccactcctcctcccctgaagtgtctgcactgtccagacccacagcctttataagtgcagggaccagccatcttctgcctcatacatcagcttctgaacaccgtcaggcatcttcacagctgcaaaggctattccacagcagaggacaatcatgagaatccttttcttccttgttgctgttctcttcttcctcttccaggctgctccaggtaagctggaaaataggtgagatggagactaaaagggcacatgcacagagaagtcctagcctctgtgctcttggactcttgtcatacttaaactccctttccttattcatagccatagtctttaacctattgagtcaaggtccttggtggtgtcatgtgaatggtggtgacatgaatgtggtgtcattctagccctgagcatcttgcgtccagttatgtgattggaaggatggaaagaagatgctctctgtgtattcaggcagctctgtgggatgacacttctgacaccatcttgtaatatgacagctttctcttgcatttacactcacatctcctgagtgtgtattctacatcagcagacctgtacttttcaaaaaatgtagttgtgactatggggcttttcctgaactttctattgtccctgtgcattaaatgggtttgaaatgcttaattctaggctattctaagtgatttagagaagtttaagctttctccgttgctgctttggtttacagcacacttgtacttcctttcctagcgcagcaagggcctgtctactcagtggagggatgtagagccacttagcccaacagcaggagcttgcaggagatcatagaatcatagaatcatagaattgctaaggttggaaaagacccacaggatcatgcagtccaaccattcgcccttcaccaatggttctcgctaaaccatgtccctcaacacaacatccaaacgctctttgaacacctccagggtcggtgactccaccacctctctgggcagcccattccagtgcctgaccaccctttcagagaagttgtatttcctaacgtccagcctgaatcttccctggcgcagcttgaagccattccctctcgtcctaacttaaatgatgtacaaagaatgcaatttgaatttcaggctgaaaatctcagtgggaatgagtaacatttaacctgtaaaccccctgatctaaacctggggccagcgtttcacatgggataagcccaggaggttgtcctggtgatgatggaattgcagtttgacatcacctgagcatttggcttcagctctgaacatatcagtcatggttaatccagcatagtggctacctaaaaccatatacagcaccagtgacaacagtcactgaccttatttcatggtattttagagctgcataagaagttctagtgggacgtgttacatcccccaggtttggctatggttaagattcgcctttcatgatttttatgccatctgttttagccttactacgttatgtttggtaacacagcttccctgtgagcatgcaggaagctattcaaatcagaagataatgagtgataaatataatttacatctgctgaaacttgagctgaaggcctgaagaagtccagttaagacattttaatttggataacatcaccttttctgggtggtgtgagattaattttaaagataccccaatcacaaacctttatgccaagcgtggtgtcaggatatgacatagcatgccttgtaatcttatgctgcatctctccttatactctgccatggtaaatgaatgtcagtttccccttgatttcttgcaccttacagccaagaagacgctgacaccttagcatgcaggcagagccacggctcctgctcttttgttgcatgccgtgctccttcagttgacattgggacctgccgtggtgggaagctgaaatgctgcaaatggtaagggagttcctcctgagaaacccaggggataaacccagcatggcataactagaagcaagtcagaactcatacctggaatacagttctccacctgggttgttctactaggtgcagagtgccaaatatcagagcaaagtgccataggagatcttccactctgtggaagtaagatgtgttttcatcagcaccatgagaaaacctctgtatctacaggagataaatagtgtggagcaatgctgtagtatgccatggtccagttctgcagctaatgacatttcatatcaaatgagctgtagcacacctacccttttttttttttggacatcataaaccggttgtttttgttttgcttatttatttatttttacttatttgcattcgtatttaattgaagtgggtttctgtgaatcactatgcagaaggataaaaaaaaaaaaaaaaaaagaataatgtgaagccttgccttgccagagccctggcacagggagcagagagccatggtggctcctcttggggatctcccacagcccctggccgtggtgctgctctgggcagccctgctggggttggggggaccagagggactcagaagtgcatccaacctcaatcattttgtgattcagtgaatatagatcagaggtccagatcacaaggaactccagtagtcgtcaacctcatgtgttaccaggtgtctacaccagcctgagatgggaaggtatcatttgcttgtctggatagagaaaggttgaataggccaccacatgatttttctttgcagggcacccagctcctaaatctgagacctcactgaccacgcagttaagagccctggaaatctgagatgtctcctgccttatgacatcactggatctttgaactttggtacaaacccaaggagcctttcccaatggggtgaagagtcctgggagcacgagaagatctgaggaggaattcttgtacctctgatacagatttgcagcttcatttctaataaaaacaattcaaagtgaaaccaca

SEQ ID NO: 4 - DNA, 18 bp, Gallus domesticus
ttctttgcagggcaccca

SEQ ID NO: 5 - DNA, 21 bp, Gallus domesticus
atttttctttgcagggcacct

SEQ ID NO: 6 - DNA, 23 bp, Gallus domesticus
atccagtgatgtcataaggcagg

SEQ ID NO: 7 - DNA, 16 bp, Gallus domesticus
ccacgcagttaagagc

SEQ ID NO: 8 - DNA, 15 bp, Gallus domesticus
cacgcagtcaagagc

SEQ ID NO: 9 - DNA, 17 bp, Gallus domesticus
tgccgtggtgggaagct

SEQ ID NO: 10 - DNA, 22 bp, Gallus domesticus
tctcagatttccagggctcttg

SEQ ID NO: 11 - DNA, 22 bp, Gallus domesticus
tctcagatttccagggctctta

SEQ ID NO: 12 - DNA, 14 bp, Gallus domesticus
atgggcacccagct

SEQ ID NO: 13 - DNA, 14 bp, Gallus domesticus
atgggcacctagct

SEQ ID NO: 14 - DNA, 20 bp, Gallus domesticus
gtggtgggaagctgaaatgc

SEQ ID NO: 15 - DNA, 24 bp, Gallus domesticus
tgatgtcataaggcaggagacatc

SEQ ID NO: 16 - DNA, 30 bp, Gallus domesticus
tcctaaatctgagacctcactgaccacgca

(19) United States
(12) Patent Application Publication
(10) Pub. No.: US 2007/0092909 A1
(43) Pub. Date: Apr. 26, 2007

(54) Molecular markers for identification of fat and lean phenotypes in chickens

(75) Inventors: Larry A. Cogburn, New London, PA (US); Wilfrid G. Carre, Edinburgh (GB); Xiaofei Wang, Nashville, TN (US)

Correspondence Address: Connolly Bove Lodge & Hutz LLP;P.O. Box 2207, 1007 North Orange Street, Wilmington, DE, 19899, US

(73) Assignee: UNIVERSITY OF DELAWARE, Newark, DE (US)

(21) Appl. No.: 11/634,641

(22) Filed: Dec. 6, 2006

Related U.S. Application Data
(63) Continuation of application No. 10/376,120, filed on Feb. 27, 2003, now abandoned.
(60) Provisional application No. 60/359,846, filed on Feb. 27, 2002.

(51) Int. Cl. C12Q 1/68

(52) U.S. Cl. 435/6

(57) ABSTRACT

The invention provides molecular methods of screening chickens to determine those more likely to have a lean or fat phenotype by identifying the presence of at least one polymorphism in genetic material of a chicken in the thyroid hormone repressible gene (THRG) or its 3′ untranslated region (SEQ ID NO: 1) that is associated with a fat phenotype or a lean phenotype. The invention also provides methods of screening chickens to identify a polymorphism associated with a fat or lean phenotype. The invention further provides oligonucleotide probes and primers useful for detecting the polymorphisms associated with a fat or lean phenotype.

This application claims the benefit of provisional application Ser. No. 60/359,846 filed Feb. 27, 2002, which is hereby incorporated by reference.

### REFERENCE TO U.S. GOVERNMENT SUPPORT

This work is supported by a grant from the USDA-IFAFS, Animal Genome Program (Award Number 00-52100-9614). The United States government has certain rights in the invention.

### FIELD OF THE INVENTION

The present invention relates to method for identifying the phenotype of a chicken using a genetic polymorphism associated with a fat or lean phenotype. More particularly the invention relates to method of identifying a fat or lean chicken phenotype by determining the presence of a polymorphism associated with a fat or lean phenotype in the thyroid hormone repressible gene (THRG).

### BACKGROUND OF THE INVENTION

Over the last decades intensive selection on growth rate has been done in broiler chicken strains developed for meat production. However, fatness has also been increased, leading to excessive adiposity. By reducing feed efficiency and lean meat yield, this excess of fat tissue is a major drawback for production. In order to decipher the metabolism and genetic mechanisms involved in the regulation of fatness in the chicken, some investigators have developed experimental models of adiposity. Lean and fat chicken lines have been divergently selected form adipose tissue weight (Leclerq et al. (1980) “Selecting broilers for low or high abdominal fat: initial observations” Br. Poul. Sci. 21, 107-113 and for very low density lipoprotein (VLDL) plasma concentration (Whitehead, C. C., Griffin, H. D., 1984. “Development of divergent lines of lean and fat broilers using plasma very low density lipoprotein concentration as selection criterion: the first three generations”. Br. Poult. Sci. 25, 573-582.) Studies performed in lean and fat lines developed by Leclercq et al (1980) indicate that the difference in adiposity between lines was not the result of a difference in food consumption or in metabolic utilization. Stearoyl-Co-A desaturase activity and plasma VLDL concentration were found to be higher in the fat line (Legrand, P. and Hermier, D., 1992. “Hepatic D9 desaturation and plasma VLDL in genetically lean and fat chickens.” Int. J. Obesity 16, 289-294), suggesting a higher lipogenesis rate in this line. In the chicken, lipogenesis occurs essentially in the liver, the adipose tissue being only a storage tissue (O'Hea, E. K and Leveille, G. A., 1968. “Lipogenesis in isolated adipose tissue of the domestic chick (Gallus domesticus)” Comp. Biochem. Physiol. 26, 111-120. 1968; Griffin et al., 1992. “Adipose tissue lipogenesis and fat deposition in leaner broiler chickens”, J. Nutr. 122,363-368.1992).

A single nucleotide polymorphism or SNP is a small genetic change or variation that can occur in an individual's DNA sequence. SNP variation occurs when a single nucleotide, such as an A, replaces one of the other three nucleotides, C, G, and T. Most SNPs are found outside of coding sequences. SNPs found within a coding sequence are of particular interest to researchers as they are more likely to alter the biological function of a protein.

Many common diseases and conditions are not caused by a genetic variation within a single gene, but are influenced by complex interactions among multiple genes as well as environmental and lifestyle factors. Genetic predisposition is the potential of an individual to develop a disease or condition based on genes and hereditary factors. Although both environmental and lifestyle factors add tremendously to the uncertainty of developing a disease it is currently difficult to measure and evaluate their overall effect on a disease process. By studying stretches of DNA that have been found to harbor a SNP associated with a disease trait, researchers may begin to reveal relevant genes associated with a disease. Researchers have found that most SNPs are not responsible for a disease state. Instead, they serve as biological markers for pinpointing a disease on a genomic map, as they are usually located near a gene found to be associated with a certain disease.

Because SNPs occur frequently throughout the genome and tend to be relatively stable genetically, they serve as excellent biological markers. A SNP associated with a disease trait can also be used as a biological marker to signal the presence of the disease in an individual or signal an increased or decreased likelihood that the individual has or will contract the disease. Therefore, it is desirable to find polymorphism(s) which can used for the diagnosis of a disease and/or identification of the trait. However, no functional polymorphisms associated with a fat or lean chicken phenotype have been reported.

### SUMMARY OF THE INVENTION

The present invention provides a molecular method of screening chickens to determine those more likely to have a lean or fat phenotype comprising the steps of obtaining a sample of genetic material from a chicken; and identifying the presence of at least one polymorphism in said genetic material in the thyroid hormone repressible gene or 3′ untranslated region as shown in SEQ ED NO: 1 that is associated with a fat phenotype or a lean phenotype. Preferably the polymorphism comprises the presence of T at the position corresponding to position 195 relative to the first base of the start codon of THRG in the coding region as shown in FIG. 3 and SEQ ID NO: 1 and the presence of C at the position corresponding to position 231 relative to the first base of the start codon of THRG which is associated with a lean phenotype, or the polymorphism comprises the presence of C at the position corresponding to position 195 relative to the first base of the start codon of THRG and T at the position corresponding to position 231 relative to the first base of the start codon of THRG which is associated with a fat phenotype.

The step of identifying the presence of the polymorphism preferably comprises the steps of amplifying a portion of the genetic material with a forward primer and a reverse primer capable of amplifying a region of the thyroid hormone repressible gene or 3′ untranslated region as shown in SEQ ID NO: 1, which region contains a polymorphic site, and detecting the polymorphism in the amplified region.

The invention also provides a method of screening chickens to identify a polymorphism associated with a fat or lean phenotype comprising obtaining a sample of genetic material from a chicken; and identifying the presence of at least one polymorphism in the genetic material in the thyroid hormone repressible gene or 3′ untranslated region as shown in SEQ ID NO: 1 that is associated with a fat phenotype or a lean phenotype.

These and other aspects of the invention are set out in greater detail in the following Detailed Description and in the accompanying claims.

### BRIEF DESCRIPTION OF THE DRAWINGS

FIG. 1 shows the consensus sequence of chicken Thyroid Hormone-Repressible Gene (THRG) contig (UD-CAP3 Contig_GP2_{—}6154) (SEQ ID NO: 1). This high-fidelity in silico cDNA sequence (596 base pairs) was assembled from 15 chicken ESTs found in public databases. The start codon ATG is shown in bold underline. The poly A site is underlined.

FIG. 2 shows the detailed alignment of 15 chicken expressed sequence tags (ESTs) and shows two single nucleotide polymorphisms (SNP1 and SNP2) in the cDNA sequence at nucleotide (nt) 195 and 231 relative to the first base of the start codon in the coding region of THRG as shown in FIG. 3A and SEQ ID NO: 1. (12 of these ESTs were sequenced at the University of Delaware).

FIG. 3A shows the structure of the THRG cDNA sequence (SEQ ID NO: 1). The 5′- and 3′-untranslated regions are shown in lower case letters. The asterisk indicates the stop codon and the polyadenlyation signal is underlined. The exon (Exon 1, Exon 2 and Exon 3) boundaries are shown by the downward arrows. The two SNP sites are shown by underlined bold letters, SNP 1 is located in the c-terminal region at nt 195 and SNP 2 is located in the 3′-untranslated region at nt 231.

FIG. 3B shows the sequence of the THRG predicted protein sequence (67 amino acids) (SEQ ID NO: 2). A thirty amino acid signal peptide is shown in bold letters. The predicted protein contains six cysteine residues that could form disulfide bonds.

FIG. 4 shows the percentage of body fat in lean phenotype and fat phenotype chicken lines as percentage of body weight. Abdominal fat content (% body weight, % BW) or growth rate of broiler chickens divergently selected for either high abdominal fat weight (fat line—FL) or low abdominal fat weight (lean line—LL) at the same body weight at nine weeks of age. Area-under-curve (AUC) values (% BW×wk) possessing a different superscript are significantly (P<0.05) different. Each value represents the average (±SEM of six birds).

FIGS. 5A-C show the segment of the chicken genomic DNA sequence (GenBank Accession number AC110874) containing THRG (SEQ ID NO:3). Exon 1—bases 188-308; Exon 2—bases 1901-2030; Exon 3—bases 2818-3100. Intronic sequence is shown in lower case letters.

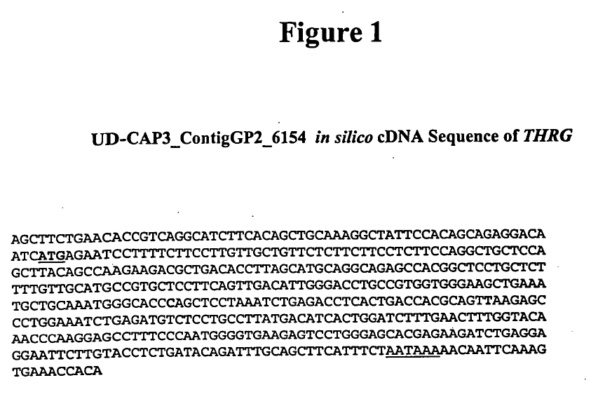

### DETAILED DESCRIPTION OF THE INVENTION

The methods of the invention are useful for identifying individual chickens or groups of chickens that have a predisposition for a lean or fat phenotype. Identification of birds having a lean or fat phenotype is of interest to chicken breeders and growers. The THRG gene and its double SNP sites are useful as a genetic marker for marker assisted selection in poultry breeding. A chicken's phenotype (lean or fat) can be determined from tissue or blood samples even before the chick is hatched, without the need for raising potential breeder chickens to adult age for measurement of the phenotype.

Applicants have found two single nucleotide polymorphisms (SNPs) in a gene referred to herein as thyroid hormone repressible gene (THRG) or the gras gene (shown in SEQ ID NO: 1) that are associated in chickens with a fat or lean phenotype.

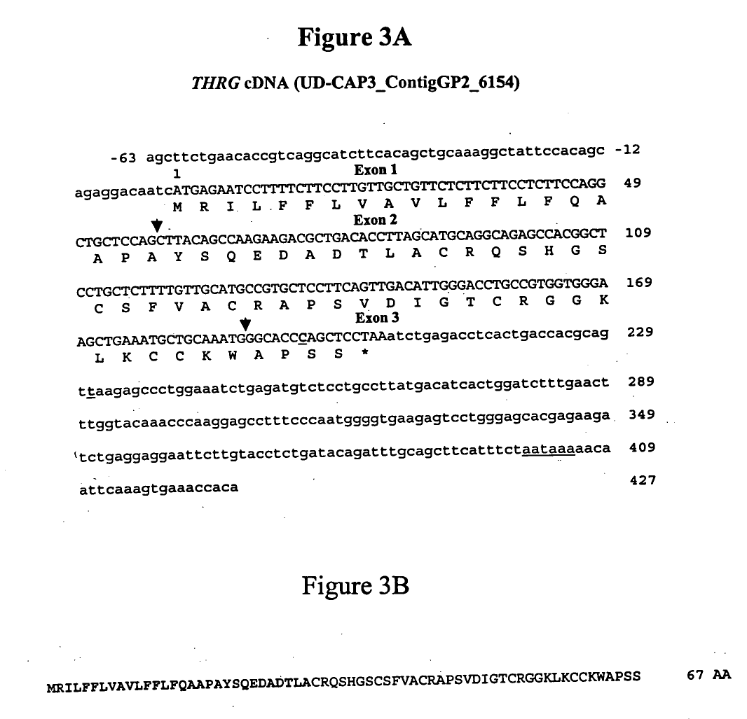

The location of the polymorphisms in THRG are given in relation to either their location in SEQ ID NO: 1, or their location relative to the start codon of the THRG protein (FIG. 3). FIG. 3 shows that the cDNA sequence (SEQ ID NO: 1) contains 63 bases upstream of the coding region, which begins at nucleotide 64. As shown in FIG. 3, with regard to SNP1, the polymorphism is at nucleotide 258 of SEQ ID NO: 1 which corresponds to nucleotide 195 relative to the first base of the start codon. With regard to SNP 2, the polymorphism is at nucleotide 294 of SEQ ID NO: 1 which corresponds to nucleotide 231 relative to the first nucleotide of the start codon. Reference herein to SNP 1 or the SNP site at nucleotide/base 195 therefore refers to nucleotide 258 of SEQ ID NO: 1 or nucleotide 195 relative to the first base of the start codon of the THRG protein. Reference herein to SNP 2 or the SNP site at nucleotide/base 231 therefore refers to nucleotide 294 of SEQ ID NO: 1 or nucleotide 231 relative to the first base of the start codon of the THRG protein.

THRG is located on chromosome 3 at 320 centi-Morgans (cM).

In the lean phenotype, the base at nucleotide 195 is T (thymidine) and the base at nucleotide 231 is C (cytosine). The SNP at nucleotide 231 is in the 3′ untranslated region of THRG. In the fat phenotype, the base at nucleotide 195 is C and the base at nucleotide 231 is T.

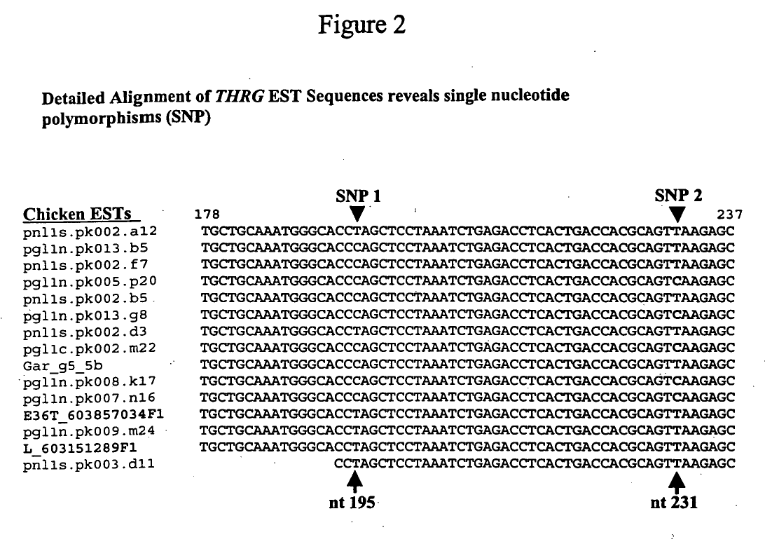

The cDNA sequence of THRG is shown in FIG. 2 (SEQ ID NO: 1). The sequence encodes a 67 amino acid protein (SEQ ID NO: 2). The exon (Exon 1, Exon 2, and Exon 3) boundaries are shown by the downward arrows. The two SNP sites are shown by bold letters. SNP 1 is located in the C-terminal region at nucleotide 195 and SNP 2 is located in the 3′ untranslated region at nucleotide 231. The predicted protein sequence (67 amino acids) (SEQ ID NO: 2) shows a thirty amino acid signal peptide in bold letters (amino acids 1-20 of SEQ ID NO: 2) and six cysteine residues that could form disulfide bonds.

In the chicken lipogenesis occurs essentially in the liver, the adipose tissue being only a storage tissue. In order to analyze genes that are differentially expressed in the liver of lean and fat chicken lines that have been divergently selected for adipose tissue weight, and to find those that play a regulatory role in adiposity, differential display analyses were performed on total RNAs extracted from the liver of lean and fat chickens. The fat and lean chicken lines used in the differential display analyses were described in (Leclercq et al, 1980. Selecting broilers for low or high abdominal fat: initial observations. Br. Poul. Sci. 21, 107-113). Among the 113 products with a differential display between the lean and fat chicken livers, 26 were selected that displayed a lean or fat specific pattern of expression or a marked difference in amplification between lean and fat animals and 23 were efficiently sequenced. One product, GAR33-G5-5B (which was found to correspond to THRG), had a significant difference between the lean and fat phenotypes (L/F=1.29). This sequence in fat and lean chicken liver RNA was then amplified and compared by sequence alignment. Two single nucleotide polymorphisms were found at position 263 and 299 of the GAR33-G5-5B sequence (corresponding to nucleotides 258 and 294, respectively, of SEQ ID NO: 1, nucleotides 195 and 231 respectively, relative to the first nucleotide of the start codon of the THRG protein), indicating the existence of two different mRNAs, a fat specific (C₂₆₃ and T₂₉₉) and a lean specific (T₂₆₃ and C₂₉₉). Single strand conformation polymorphism (SSCP) analyses were then performed on a larger sample of chickens from the lean and fat lines and from R+ and R− egg laying lines. A “lean specific band” was present in most animals from the lean and R+ lines and a different “fat specific band” which had a lower position on the SSCP electrophoresis gel than the “lean specific band” was present in fat and R− animals. Two lean animals were found to have an unexpected “fat specific band” and in some animals, especially those from the lean line, a more complex pattern was observed corresponding to both the “lean and fat specific” products.

Further sequence analysis and analysis of EST sequences showed that the polymorphisms were located at positions 258 and 294 of the consensus sequence of THRG contig (UD_CAP3 contig_GP_{—}6154) which is shown in SEQ ID NO: 1.

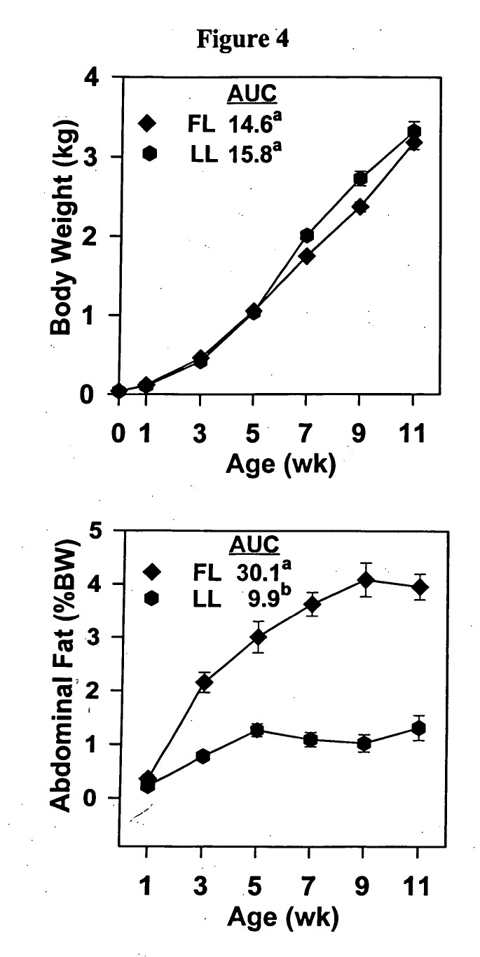

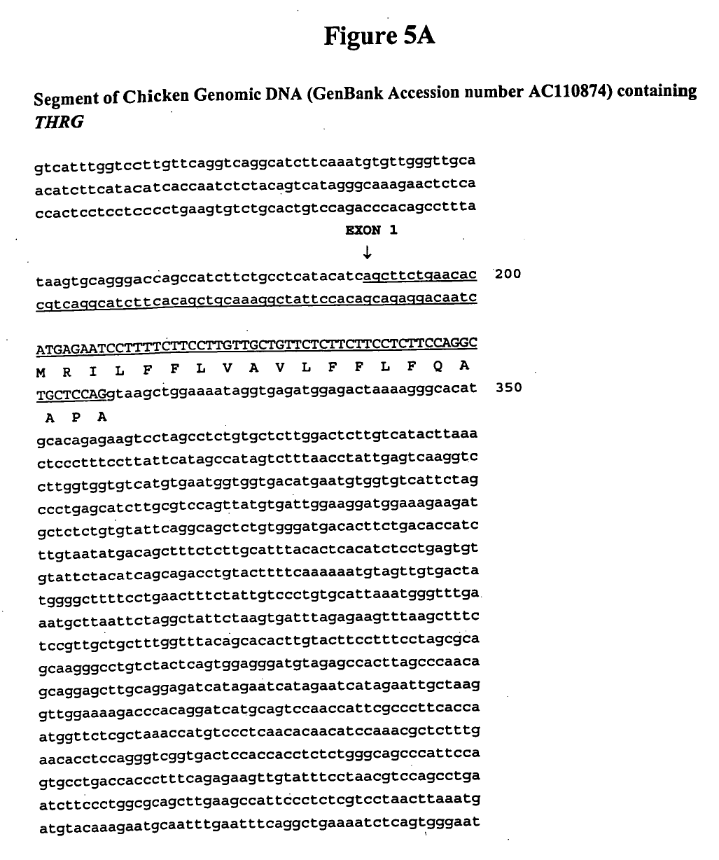

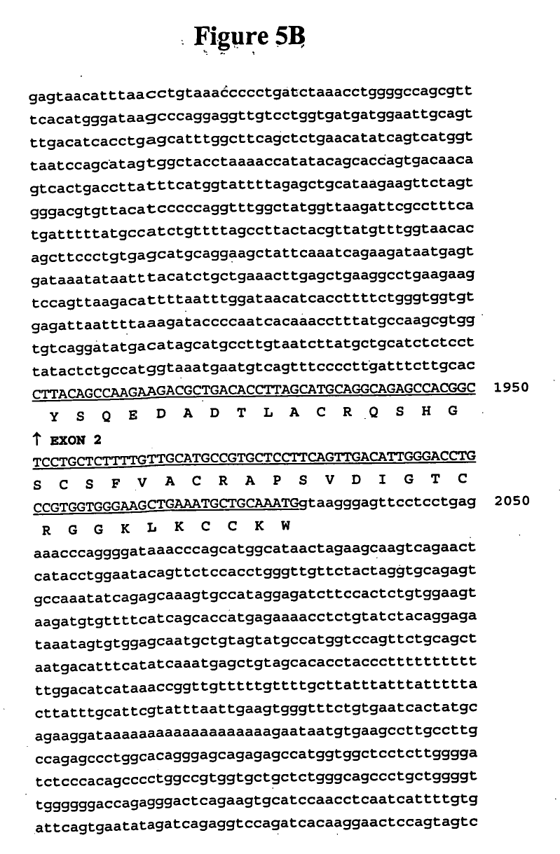

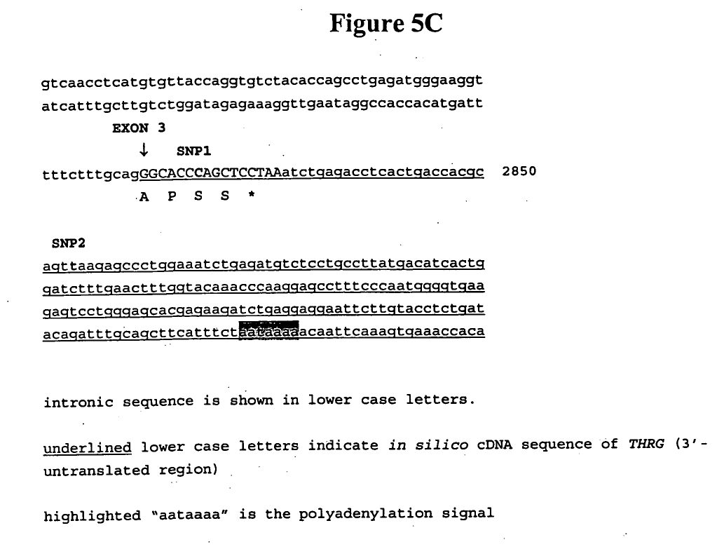

Fat phenotype and lean phenotype refer to the phenotypes of the lean and fat lines of chickens developed by Leclerq et al. 1980. British Poultry Science 21: 107-113. Birds having a fat phenotype have about three to four times as much abdominal fat as birds having a lean phenotype, as shown in FIG. 4. Abdominal fat is measured by measuring the live body weight (in g or kg), killing the bird, careful dissection of the abdominal fat pad including that surrounding the ventriculus (gizzard) and that surrounding the cloaca (rectum), then measuring the weight of the dissected abdominal fat pad, and is expressed as percent of body weight (% BW). In FIG. 4, FL refers to the fat line of chickens that have the fat phenotype. LL refers to the lean line of chickens that have the lean phenotype.

Fat phenotype thus refers to a phenotype wherein abdominal fat is about 3-4% of body weight. Lean phenotype refers to a phenotype wherein abdominal fat is about 1 to 1.2% of body weight. However, Whitehead, C. C., Griffin, H. D. (1984) have divergently selected lean and fat lines of chickens based on low or high plasma very low density lipoprotein (VLDL) levels, respectively. These fat and lean lines of chickens differ in their abdominal fat content (g/kg BW) by only 49%. Thus, the degree of leanness or fatness selected in a given population of chickens could vary depending on the genetic background and the selection criteria. Therefore, the definition of leanness or fatness should be based on a phenotypic difference in the average abdominal fat content (% BW) with a difference of least two standard error units.

The present invention thus provides molecular methods for screening chickens to determine those more likely to have a lean or fat phenotype comprising the steps of obtaining a sample of genetic material from a chicken; and identifying the presence of at least one polymorphism in genetic material in the thyroid hormone repressible gene or its 3′ untranslated region as shown in SEQ ID NO: 1 that is associated with a fat phenotype or a lean phenotype. Preferably, the at least one polymorphism comprises the presence of T at the position corresponding to position 195 and the presence of C at the position corresponding to position 231 which is associated with a lean phenotype, or the least one polymorphism comprises the presence of C at the position corresponding to position 195 and T at the position corresponding to position 231 which is associated with a fat phenotype.

The invention also provides methods of screening chickens to identify a polymorphism associated with a fat or lean phenotype comprising obtaining a sample of genetic material from a chicken; and identifying the presence of at least one polymorphism in said genetic material in the thyroid hormone repressible gene or 3′ untranslated region as shown in SEQ ID NO: 1 that is associated with a fat phenotype or a lean phenotype. Preferably, the at least one polymorphism comprises the presence of T at the position corresponding to position 195 and the presence of C at the position corresponding to nucleotide 231 which is associated with a lean phenotype, or the least one polymorphism comprises the presence of C at the position corresponding to position 195 and T at the position corresponding to position 231 which is associated with a fat phenotype.

Genetic material used in the methods of the invention may be isolated from cells, tissues, blood or other samples according to standard methodologies, such as the methods in Sambrook et al., Molecular Cloning: A Laboratory Manual, Cold Spring Harbor Laboratory Press, Cold Spring Harbor, N.Y., (1989). In certain embodiments, analysis is performed on whole cell or tissue homogenates or biological fluid samples without substantial purification of the template nucleic acid. The genetic material may be genomic DNA or RNA. Where RNA is used, it may be desired to first convert the RNA to a complementary DNA. A preferred source of genetic material is blood. Chickens have nucleated red blood cells which makes blood a convenient source of genetic material.

The polymorphisms indicative of a fat or lean phenotype can be identified by any method known in the art for detection of alleles at specific polymorphic sites. Suitable methods include sequencing the genetic material, polymerase chain reaction (PCR)-based assays, primer extension, and allele-specific oligonucleotide ligation.

A number of template dependent processes are available to amplify the oligonucleotide sequences present in a given template sample. One of the best-known amplification methods is the polymerase chain reaction (referred to as PCR) which is described in detail in U.S. Pat. Nos. 4,683,195, 4,683,202 and 4,800,159, each of which is incorporated herein by reference in their entirety.

A reverse transcriptase PCR (RT-PCR) amplification procedure may be performed to quantify the amount of mRNA amplified. Methods of reverse transcribing RNA into cDNA are well known and described in Sambrook et al., Molecular Cloning: A Laboratory Manual, Cold Spring Harbor Laboratory Press, Cold Spring Harbor, N.Y., (1989). Alternative methods for reverse transcription utilize thermostable DNA polymerases. These methods are described in WO 90/07641. Polymerase chain reaction methodologies are well known in the art. Representative methods of RT-PCR are described in U.S. Pat. Nos. 5,882,864, 5,673,517 and 5,561,058.

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single-strand conformation polymorphism analysis (“SSCP”) and other methods well known in the art.

The amplification product may be detected or quantified. In certain applications, the detection may be performed by visual means. Alternatively, the detection may involve indirect identification of the product via chemiluminescence, radioactive scintigraphy of incorporated radiolabel or detection of a fluorescent label.

A preferred method for detecting the SNPs is real-time quantitative PCR using dual labeled TaqMan® probes which have a fluorophore at the 5′ end and a quencher at the 3′ end. Methods for performing PCR using dual labeled probes are disclosed in U.S. Pat. Nos. 5,210,015, 5,804,375, 5,487,792 and 6,214,979.

PCR technology relies on thermal strand separation followed by thermal dissociation. During this process, at least one primer per strand, cycling equipment, high reaction temperatures and specific thermostable enzymes are used (U.S. Pat. Nos. 4,683,195 and 4,883,202). Alternatively, it is possible to amplify the DNA at a constant temperature (Nucleic Acids Sequence Based Amplification (NASBA) Kievits, T., et al., J. Virol Methods, 1991; 35, 273-286; and Malek, L. T., U.S. Pat. No. 5,130,238; and Strand Displacement Amplification (SDA), Walker, G. T. and Schram, J. L., European Patent Application Publication No. 0 500 224 A2; Walker, G. T., et al., Nuc. Acids Res., 1992; 20, 1691-1696; and the like). Any sequencing method known to a person skilled in the art may be employed. In particular, it is advantageous to use an automated DNA sequencer. The sequencing is preferably carried out with a double-stranded template by means of the chain-termination method using fluorescent primers. An appropriate kit for this purpose is provided from PE Applied Biosystems (PE Applied Biosystems, Norwalk, Conn., USA).

The single strand conformation polymorphism (SSCP) detection technique is a method involving separation on an acrylamide gel, but under non-denaturing conditions. It is performed preferably with capillary electrophoresis equipment. This technique makes it possible to discriminate between different DNA fragments in terms of their conformation.

Alternatively, the DNA chip method can be employed (Barinaga M., Science, 1991; 253, 1489; Bains, W., Bio/Technology, 1992; 10, 757-758; Wang et al., Science, 1998; 280, 1077-1082). These methods usually attach specific DNA sequences to very small specific areas of a solid support, such as micro-wells of a DNA chip. Each type of polymorphic DNA of the present invention can be used for the DNA chip when they are hybridized with the amplified DNA fragment of the genetic material sample, and then detected by the pattern of hybridization.

The polymorphisms can also be identified by hybridization to nucleic acid arrays, some examples of which are described in WO 95/11995. The same arrays or different arrays can be used for analysis of characterized polymorphisms. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

Alleles of target sequences can be differentiated using single-strand conformation polymorphism (SSCP) analysis, which identifies base differences by alteration in electrophoretic migration of single stranded PCR products (Orita et al., 1989. Proc. Nat. Acad. Sci. 86:2766-2770). Amplified PCR products can be generated as described above, and heated or otherwise denatured, to form single-stranded amplification products. Single-stranded nucleic acids may re-fold or form secondary structures that are partially dependent on the base sequence. The different electrophoretic mobilities of single-stranded amplification products can be related to base-sequence differences between alleles of target sequences.

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., 1997. PNAS 94:10756-61, uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs). An increase in fluorescence of the added ddNTP in response to excitation at the wavelength of the labeled primer is used to infer the identity of the added nucleotide. Other suitable methods will be readily apparent to the skilled artisan.

The invention also provides primers and probes for use in the assays to detect the SNPs. The primers and probes are based on and selected from SEQ ID NO: 1 and will typically span the region of SEQ ID NO: 1 upstream or downstream of a SNP in the case of primers, or span a SNP site in the case of a probe and will have a length appropriate for the particular detection method. The primers and/or probes can also be based on and selected from the genomic DNA sequence of THRG (SEQ ID NO: 3). One aspect of the invention thus provides oligonucleotides comprising from about 10 to about 30 contiguous bases of SEQ ID NO: 1 or SEQ ID NO: 3, or the complementary sequence of SEQ ID NO: 1 or SEQ ID NO: 3 for use as probes or primers.

Probes can be any length suitable for specific hybridization to the target nucleic acid sequence. The most appropriate length of the probe may vary depending upon the hybridization method in which it is being used; for example, particular lengths may be more appropriate for use in microfabricated arrays (microarrays), while other lengths may be more suitable for use in classical hybridization methods. Such optimizations are known to the skilled artisan. Suitable probes can range from about 5 nucleotides to about 30 nucleotides in length. For example, the probes can be 5, 6, 8, 10, 12, 14, 16, 18, 20, 22, 24, 25, 26, 28 or 30 nucleotides in length. Additionally, a probe can be a genomic fragment that can range in size from about 25 to about 2,500 nucleotides in length. The probe preferably overlaps at least one polymorphic site occupied by any of the possible variant nucleotides. The nucleotide sequence of the probe can correspond to the coding sequence of the allele or to the complement of the coding sequence of the allele.

Preferably, the PCR probes are TaqMan® probes which are labeled at the 5′end with a fluorophore, and at the 3′-end with a quencher or a minor groove binder and a quencher (for minor groove binding assays). Suitable fluorophores and quenchers for use with TaqMan® probes are disclosed in U.S. Pat. Nos. 5,210,015, 5,804,375, 5,487,792 and 6,214,979.

An oligonucleotide primer can be synthesized by selecting any continuous 10 to 30 base sequence from the sequence of SEQ ID NO: 1 or the complementary sequence of SEQ ID NO: 1. The length of these oligonucleotide primers are commonly in the range of 10 to 30 nucleotides in length, preferably in the range of 18 to 25 nucleotides in length.

Hybridizations can be performed under stringent conditions, e.g., at a salt concentration of no more than 1 M and a temperature of at least 25.degree. C. For example, conditions of 5×SSPE (750 mM NaCl, 50 mM Na-Phosphate, 5 mM EDTA, pH 7.4) and a temperature of 25-30.degree. C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

The reaction mixture for amplifying the DNA comprises 4 deoxynucleotide phosphates (dATP, dGTP, dCTP, dTTP) and heat stable DNA polymerase (such as Taq polymerase), which are all known to the skilled person in the art.

Applicants discovered that the genomic THRG DNA sequence contains introns near to the SNP site at base 195. The genomic DNA sequence of THRG (SEQ ID NO: 3) contains introns near the SNP site at base 195. Depending on the methods used to detect the SNPs, it may be necessary to use different primer/probe sets to detect the SNPs in genomic DNA or RNA.

A preferred primer/probe set for detection of the SNPs in genomic DNA is:

| | |
|---|---|
| TTCTTTGCAGGGCACCCA; | (SEQ ID NO: 4 |
| ATTTTTCTTTGCAGGGCACCT; | (SEQ ID NO: 5) |
| ATCCAGTGATGTCATAAGGCAGG; | (SEQ ID NO: 6) |
| 6FAM-CCACGCAGTTAAGAGC- | (SEQ ID NO: 7) |
| CACGCAGTCAAGAGC | (SEQ ID NO: 8) |

A preferred primer probe set for detection of the SNPs in RNA/cDNA is:

| | |
|---|---|
| TGCCGTGGTGGGAAGCT; | (SEQ ID NO: 9) |
| TCTCAGATTTCCAGG GCT CTT G; | (SEQ ID NO: 10) |
| TCTCAGATTTGCAGGGCTCTT A; | (SEQ ID NO: 11) |
| ATGGGCACCCAGCT; | (SEQ ID NO: 12) |
| ATG GGC ACC TAG CT | (SEQ ID NO: 13) |

A preferred primer set for detection of THRG RNA/cDNA is:

| | |
|---|---|
| GTGGTGGGAAGCTGAAAT GC; | (SEQ ID NO: 14) |
| TGATGTCATAAGGCAGGAGACATC; | (SEQ ID NO: 15) |
| TCCTAAATCTGAGACCTCACTGACCACGCA. | (SEQ ID NO: 16) |

Because of the close proximity of the SNPs in the THRG nucleotide sequence, it is necessary, when detecting the SNPs using PCR and dual labeled TaqMan probes, to detect each SNP separately. The preferred primer/probe sets thus contain a set of primers and probes to detect the polymorphism at nucleotide 195, and a set of primers and probes to detect the polymorphism at nucleotide 231. The preferred primer and probe sets are described in more detail in the examples.

The oligonucleotide primers and probes can be synthesized by any technique known to a person skilled in the art, based on the structure of SEQ ID NO: 1 or SEQ ID NO: 3.

The invention further provides kits comprising at least one allele-specific oligonucleotide or gene expression product indicator as described herein. Often, the kits contain one or more pairs of allele-specific oligonucleotides hybridizing to different forms of a polymorphism. Examples of suitable allele-specific oligonucleotides include the oligonucleotide probes disclosed herein. The kits can also comprise primers for amplifying a region of SEQ ID NO: 1 or SEQ ID NO: 3 that spans a polymorphism. Optionally, the allele-specific oligonucleotides are provided immobilized to a substrate. The assay kit can further comprise the four deoxynucleotide phosphates (dATP, dGTP, dCTP, dTTP) and an effective amount of a nucleic acid polymerizing enzyme. A number of enzymes are known in the art which are useful as polymerizing agents. These include, but are not limited to E. coli DNA polymerase I, Klenow fragment, bacteriophage T7 RNA polymerase, reverse transcriptase, and polymerases derived from thermophilic bacteria, such as Thermus aquaticus. The latter polymerases are known for their high temperature stability, and include, for example, the Taq DNA polymerase I. Other enzymes such as Ribonuclease H can be included in the assay kit for regenerating the template DNA. Other optional additional components of the kit include, for example, means used to label the probe and/or primer (such as a fluorophore, quencher, chromogen, etc.), and the appropriate buffers for reverse transcription, PCR, or hybridization reactions. Usually, the kit also contains instructions for carrying out the methods.

The invention further provides an isolated polynucleotide comprising at least the coding portion of SEQ ID NO: 1. The isolated polynucleotide can also comprise the 3′ untranslated region of SEQ ID NO: 1. The isolated polynucleotide can be DNA or RNA.

The polynucleotide can be of the invention can be made with standard molecular biology techniques known in the art and disclosed in manuals such as Sambrook et al., Molecular Cloning: A Laboratory Manual, Cold Spring Harbor Laboratory Press, Cold Spring Harbor, N.Y., USA (1989). Synthetic chemistry techniques can be used to synthesize polynucleotides encoding antibodies of the invention

The invention also provides an isolated genomic DNA sequence comprising SEQ ID NO: 4.

The invention additionally provides an isolated polypeptide encoded by SEQ ID NO: 1. Preferably the isolated polypeptide comprises SEQ ID NO: 2. The polypeptide can be synthesized using recombinant DNA technology or chemical methods to synthesize its amino acid sequence. Suitable chemical methods include direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer).

### BRIEF DESCRIPTION OF THE NUCLEOTIDE SEQUENCES IN THE SEQUENCE LISTING

SEQ ID NO: 1—Consensus sequence of chicken Thyroid Hormone-Repressible Gene (THRG) contig (UD-CAP3 Contig_GP2_{—}6154) and coding region.

SEQ ID NO: 2—THRG predicted protein

SEQ ID NO: 3—THRG genomic DNA sequence (Segment of Chicken Genomic DNA (GenBank Accession Number AC110874) that contains THRG)

SEQ ID NO: 4—Forward primer

SEQ ID NO: 5—Forward primer

SEQ ID NO: 6—Reverse primer

SEQ ID NO: 7—Probe

SEQ ID NO: 8—Probe

SEQ ID NO: 9—Forward primer

SEQ ID NO: 10—Reverse primer

SEQ ID NO: 11—Probe

SEQ ID NO: 12—Probe

SEQ ID NO: 13—Probe

SEQ ID NO: 14—Forward primer

SEQ ID NO: 15—Reverse primer

SEQ ID NO: 16—Probe

All patents and patent applications cited in the present application are expressly incorporated herein by reference for all purposes. The above disclosure generally describes the present invention. A more complete understanding can be obtained by reference to the following specific examples, which are provided for purposes of illustration only and are not intended to limit the scope of the invention.

### EXAMPLE 1

### Identification of SNPs in THRG

Materials and Methods

Cell Culture

Chicken hepatoma LMH cells (Kawaguchi et al., 1987. Establishment and characterization of a chicken hepatocellular carcinoma cell line, LMH. Cancer Res. 47, 4460-4464) were cultured in Williams' E medium as previously described (Lefevre et al., 2001, “Effects of polyunsaturated fatty acids and clofibrate on chicken stearoyl-CoA desaturase 1 gene expression”, Biochem. Biophys. Res. Commun. 280, 25-31). One day before the experiments, cells were cultured without serum. Cells were then incubated for 6 h with 10% (v/v) fetal calf serum (FCS), 1 mM insulin, 1.5 mM triiodothyronine (T3), 50 mM eicosatetraïonic acid (ETYA) or 1 mM dexamethasone (Dexa) before RNA extraction. Control cells were incubated without serum and effectors. HepG2 cells (Knowles et al., 1980 Human hepatocellular carcinoma cell lines secrete the major plasma proteins and hepatitis B surface antigen. Science 209, 497-499) were cultured in Williams' E medium with 10% FCS.

RNA Extraction RNAs were extracted according to the method of Chomczynski and Sacchi (1987) Single-step method of RNA isolation by acid guanidium thiocyanate-phenol-chloroform extraction. Anal. Bio-chem.162, 156-159, from the liver of lean (L) and fat (F) male chickens (Gallus domesticus) divergently selected for high and low abdominal fat content (Leclercq et al., 1980, supra) and of R+ and R− egg-laying lines divergently selected for residual food intake (Bordas et al., 1992 Direct and correlated responses to divergent selection for residual food intake in Rhode Island Red laying hens. Br. Poul. Sci. 33, 741-754) as previously described (Daval et al., 2000 —Messenger RNA levels and transcription rates of hepatic lipogenesis genes in genetically lean and fat chickens”. Genet. Sel. Evol. 32, 521-531; Lagarrigue et al., 2000 Hepatic lipogenesis gene expression in two experimental egg-laying lines divergently selected on residual food consumption. Genet. Sel. Evol. 32, 205-216), from different tissues from a commercial chicken and from LMH hepatoma cells and chicken hepatocytes in primary culture as previously described (Diot and Douaire, 1999 Characterization of a cDNA sequence encoding the peroxisome proliferator activated receptor a in the chicken. Poul. Sci. 78, 1198-1202; Lefevre et al., 2001, supra). RNAs were also extracted from confluent HepG2 cells.

Differential Display Analyses

Differential display (DD) analyses were performed according to the method of Liang and Pardee (1992) “Differential display of eukaryotic messenger RNA by means of the polymerase chain reaction”. Science 257, 967-971, and Welsh et al. (1992) —Arbitrary primed PCR fingerprinting of RNA”. Nucl. Acids Res. 20, 49654970, and isolation of DD products as previously described (Carré et al., 2001 “Development of 112 unique expressed sequence Tags from chicken liver using an arbitrarily primed reverse transcriptase-polymerase chain reaction and single strand conformation gel purification method”. Anim.Genet. 32, 289-297). Total RNAs (200 ng) were extracted from the liver of five lean and five fat chickens and analyzed by differential display, individually and after pooling in two different lean and fat RNA pools using L2(T)₁₂ G or L2(T)₁₂ C reverse-primers and one of the L1AP1-L1AP6 arbitrary forward-primers (Carré et al., 2001, supra).

Specific PCR Amplification of Purified Products

Amplifications of some purified products were performed using specific primers selected from the sequence of DD products using the Primer3 software (Rozen and Skaletsky, 2000,—Primer3 on the WWW for general users and for biologist programmers”. Methods Mol. Biol. 132, 365-386.).

Sequencing of Amplified Products and Sequence Analyses

Amplified products were sequenced as described in Carré et al., 2001 supra) and sequence analyses performed essentially with BLASTN 2.2.1 and TBLASTX 2.2.1 (Altschul et al., 1997 —Gapped BLAST and PSI-BLAST: a new generation of protein database search programs”. Nucl. Acids Res. 25, 3389-3402.; against public sequence databases. Multiple sequence alignments were performed with MultAlin (Corpet, 1988 “Multiple sequence alignment with hierarchical clustering”. Nucl. Acids Res. 16, 10881-10890; and Clustal W (Thompson et al., 1994, —CLUSTAL W: improving the sensitivity of progressive multiple sequence alignment through sequence weighting, position-specific gap penalties and weight matrix choice”. Nucl. Acids Res. 22, 4673-4680; and sequence assembling with CAP3 (Huang and Madan, 1999, “CAP3: A DNA sequence assembly program”, Genome Res. 9, 868-877).

Northern Blot Analyses of Differential Products

RNA levels were analyzed by Northern blot as described in (Lefevre et al., 2001, supra) using probes corresponding to ³²P-labelled differential products. Hybridizations were revealed using a Storm™ 840 (Amersham Pharmacia Biotech) to scan exposed phosphor screens. RNA levels were expressed as relative units or as a percent of control and after correction by 18S rRNA level.

Results

Differential Display and Sequence Analyses

Among the 113 products with a differential display between the lean and fat chicken livers, 26 were selected that displayed a lean or fat specific pattern of expression or a marked difference in amplification between lean and fat animals and 23 were efficiently sequenced. Ten showed sequence similarity with mammalian sequences of nine identified genes involved in functions including translation (translation elongation factor 1 delta and ribosomal L9 and L31 proteins) and metabolism (monocarboxylate transporter SLC16A1, thyroid hormone responsive spot 14, cytochrome P450 sub-family member and NADH dehydrogenase subunit V). The 13 remaining sequences, corresponding to 11 unrelated sequences, did not match with identified gene sequences in public databases.

Northern Blot Analyses of Differential Expression

Total RNA was extracted from the liver of 12 lean chickens and organized in three different ‘lean RNA pools’. The same was done with 15 fat chickens resulting in three different ‘fat RNA pools’. RNA levels were determined by Northern blot analyses with the 26 selected DD products as radiolabelled probes. Some probes (6/26) gave a weak hybridization signal and/or near the limit of detection and RNA levels could not be determined. Among the genes that were revealed with a convenient hybridization signal (20/26), most of them (15/20) were expressed with no significant difference between lean (L) and fat (F) chickens (T-test probability, P≧0.05). However, differences were found to be significant (P<0.05) for five DD products (Table 1). Three of these showed sequence similarities with identified mammalian genes in public databases. They corresponded to CGI-109 protein (F/L=1.42) whose function remains unknown, thyroid hormone responsive spot 14 (THRSP, L/F=1.17) involved in the regulation of lipid metabolism (Cunningham et al., 1998, “‘Spot 14’ protein: a metabolic integrator in normal and neoplastic cells, Thyroid 8, 815-825), and a chicken orthologue of a mammalian cytochrome P450 2C (CYP2C ) subfamily member (L/F=3.91), including four genes in mammals that metabolize foreign chemicals as well as a number of endogenous compounds (Goldstein and de Morais, 1994). This chicken gene was recently described as CYP2C45 (Baader et al., 2002). Two DD products did not match with identified gene sequences in public databases and corresponded to GAR33-G5-5B (L/F=1.29) and GAR25-G3-8A (L/F=1.29) products. The difference between L and F RNA level was found to be nearly significant (P=0.068 and 0.072, depending on the probe used) for a gene corresponding to translation elongation factor 1 delta (EIF4A2 ) with L/F ratio of 1.92 or 2.18, depending on the experiment and/or the probe used. Most of the differential genes analyzed were found to have low differential expression, corresponding to L/F or F/L ratio ranging from 1.17 to 1.42 (depending on the probe used).

Analyses of Differential Display Products with Structural Differences

When total RNAs extracted from the liver of L and F chickens were analyzed by Northern blot analysis with the GAR120-C6-2C (C6-2C) probe, a 0.8 kb RNA was observed with a difference in length (around 50 bp) between lean and fat chickens (FIG. 2A). Similar results were observed on RNAs prepared from the liver of other L or F chickens analyzed individually, and with GAR33-G5-5B and X6-1A probes, which are similar in sequence to GAR120-C6-2C (Carré et al., 2001, supra). As observed by DD analysis, GAR120-C6-2C (C6-2C) and X6-1A products were predominantly displayed in fat chicken RNAs (F) whereas the GAR33-G5-5B (G5-5B) product was predominantly displayed in lean chicken RNAs (L).

Specific primers from the G5-5B sequence (forward 5-CCAGCAGAGGACAAT-CATGA-3 and reverse 5-CAGTGATGTCATAAGGCAGG-3) and used them to amplify lean and fat chicken liver RNAs by RT-PCR. The sequences of these RT-PCR products (RTL and RTF, respectively) were compared with the sequences of the three differential products above by multiple sequence alignments. Two single nucleotide polymorphisms were found at position 263 and 299 indicating the existence of two different mRNAs, a fat specific (C263 and T299) and a lean specific (T263 and C299). Single strand conformation polymorphism (SSCP) analyses were performed on RT-PCR products prepared with G5-5B specific primers from a larger sample of chickens from the lean and fat lines and from the R+ and R− egg-laying lines. A ‘lean specific band’ was present in most animals from the lean and R+ lines and a lower ‘fat specific band’ was present in fat and R− animals. Only two lean animals (Lean 10 and R+8) were found with an unexpected ‘fat specific band’. In some animals, and especially in those from the lean line (Lean 1, 6, 7, 8, 9,11, 12, 13 and 14, and R+2), a more complex pattern was observed corresponding to both the ‘lean and fat specific’ products. Additional bands were observed in some animals. The data indicate that ‘lean and fat specific products’ were amplified from liver RNAs extracted from a larger sample of lean and fat birds from either the same broiler lines or from divergent egg-laying lines.

Tissue Distribution of G5-5B Product and Regulation of Expression

Expression of the polymorphic RNA was analyzed in different tissues by Northern blot with the G5-5B probe. The RNA was expressed at a very low level in heart, lung, adipose tissue, kidney, duodenum, uropygial gland, muscle and brain. However, high RNA levels were found in the liver and in cultured liver cells, i.e. hepatocytes in primary culture and LMH cells. These data indicate a liver specific expression of the corresponding gene. In order to determine the function of this gene, regulation of its expression was further analyzed in LMH cells, incubated in the presence of serum, insulin, T3 or ETYA. When compared to control cells incubated without serum and effector (C), insulin (Ins) was shown to be without effect. Conversely, serum (FCS) slightly induced G5-5B expression whereas T3 and ETYA down regulated it. Expression of G5-5B was not observed in human hepatoblastoma HepG2 cells. A marked increase (about 7-fold) in RNA level was observed when cells were cultured in the presence of Dexa compared to control cells. The data also suggested that the induced RNA is greater in length than that observed in control cells.

In Silico Analyses and cDNA Cloning of G5-5B and Related Products

In order to obtain more information about the sequence and function of G5-5B and related products, some in silico analyses were first performed. TC6809, a 591 bp long TIGR Gallus gallus Gene Index cluster (GgGI version 2.0, http://www.tigr.org/tdb/gggi/) was found, assembling all G5-5B related expressed sequence tags (ESTs) deposited in public databases, and with the addition of 15 and 104 nucleotides at the 5 and 3 ends, respectively. The cluster sequence and translated products were compared to sequences in public databases. No match was found with identified genes or protein. Open reading frames (ORF) present in the sequence were also analyzed for the presence of functional motifs. Again, no probant identification was obtained.

Discussion

A sequence polymorphism was found by SSCP gel electrophoresis and sequencing between C6-2C and X6-1A products selected from fat chicken livers and the G5-5B product selected from lean chicken livers. SSCP analyses also indicated that these products are also found—as expected—in R+ and R− egg-laying hen lines, with the ‘fat product’ in R− chickens and the ‘lean product’ in R+ chickens. Previous studies have indicated that the R− line was fatter than the R+ line (El-Kazzi et al., 1995, “Divergent selection of residual food intake in Rhode Island Red egg-laying lines: gross carcase composition, carcase adiposity and lipid content of Tissues”. Br. Poul. Sci. 36, 719-728.). Furthermore, as observed by Northern blot analyses, this sequence polymorphism is associated with a greater difference in RNA length. Northern blot analyses also indicated that T3 and ETYA down-regulate and that dexamethasone strongly increases expression of the gene, while insulin has no effect. T3, ETYA and dexamethasone are activators of different nuclear receptors with similar direct repeat response elements. We can speculate that these activated factors are in competition for the same response element. This competition could also include the hepato-specific nuclear receptor HNF4, due to the liver specific expression of G5-5B gene.

Further sequence analysis and analysis of EST sequences showed that the polymorphisms were located at positions 258 and 294 of the consensus sequence of THRG contig (UD_CAP3 contig_GP_{—}6154) which is shown in SEQ ID NO: 1.

### EXAMPLE 2

### Quantitative RT-PCR for Detection of RNA Expression Levels

For each sample to be tested, the reaction mixture is prepared by combining appropriate amounts of reagents shown in [1]. The reaction mixture is placed into wells of a 384-well plate and the plate is processed by the ABI Prism® 7900HT Sequence Detection System (Applied Biosystems, Foster City, Calif., USA). The parameters of thermocycler are set as indicated by the instruction manual supplied by the manufacturer. Data are collected and analyzed using the Sequence Detection System (SDS) software (Applied Biosystems) to get a Ct value, where Ct is the number of PCR cycles required to reach the detection threshold. The Ct value is converted into the absolute amount of RNA template in the test sample, where smaller Ct values represent higher mRNA levels.

[1] Q RT-PCR reaction mixture (Quantitect ®Sybr-Green RT-PCR
Kit (Cat. # 204243); QIAGEN Inc., Valencia California, USA)

| | | |
|---|---|---|
| Forward primer: | 0.5 | μM |
| Reverse primer | 0.5 | μM |
| RT-PCR master mix | 10 | μl |
| Reverse transcriptase | 0.2 | μl |
| Total RNA | 100 | ng |
| H₂0 q.s. | 20 | μl |

Thermocycler settings are as follows: 50° C. 20 min, 1 cycle; 95° C. 15 min, 1 cycle; 90° C. 15 sec; and 60° C. 60 sec, 60 cycles.

The following primers and probe are used in the assay:

| | | |
|---|---|---|
| Forward Primer: | GTG GTG GGA AGC | (SEQ ID NO: 14) |
| | TGA AAT GC | |
| Reverse Primer: | TGA TGT CAT AAG | (SEQ ID NO: 15) |
| | GCA GGA GAC ATC | |
| Probe: | Universal | |
| | Sybr-Green | |

### EXAMPLE 3

### Allele Detection for DNA Using TaqMan® Minor Groove Binding Assay

For each DNA sample to be tested, two reaction mixtures are prepared by combining appropriate amount of reagents shown in [2] and [3]. The reaction mixtures are placed into separate wells of a 384-well plate and the plate is processed by the ABI Prism® 7900HT sequence detection system (Applied Biosystems). The thermocycler parameters are set as indicated by the manufacturer's instructions. When the thermocycler is done, data collected are analyzed using the Sequence Detection System (SDS) software (Applied Biosystems) to determine if the reaction is positive or negative for a particular base substitution. Detection of fluorescence from 6FAM indicates the presence of T at the SNP; fluorescence from VIC indicates the presence of C at the SNP. The genotype is then assigned to the tested sample based on either a positive or negative reaction, according to the table of genotype calls below. In the genotype column, the first pair of letters refers to the SNP at nucleotide 195 and 231, respectively, of one strand of DNA, and the second pair of letters refer to the SNP at nucleotides 195 and 231, respectively, in the other strand of DNA. A+ indicates a positive reaction. A negative reaction is indicated by a blank.

[2] PCR Reaction Mixture

| | | |
|---|---|---|
| Reverse primer | 0.5 | μM |
| Forward C primer | 0.1 | μM |
| Probes: C and T | 0.1 | μM |
| PCR master mix | 10 | μl |
| DNA | 50 | ng |
| H₂0 q.s. | 20 | μl |

(The PCR Master Mix is TaqMan® Universal PCR Master Mix Kit (#4304437); Applied Biosystems.)

[3] PCR Reaction Mixture

| | | |
|---|---|---|
| Reverse primer | 0.5 | μM |
| Forward T primer | 0.1 | μM |
| Probes: C and T | 0.1 | μM |
| PCR master mix | 10 | μl |
| DNA | 50 | ng |
| H₂0 q.s. | 20 | μl |

(The PCR Master Mix is TaqMan(D Universal PCR Master Mix Kit (#4304437); Applied Biosystems.)

Thermocycler Settings are as follows: 95 C° 15 min, 1 cycle; 90 C° 15 sec; 60 C° 60 sec, 25 cycles.

The following probes and primers are used in the assay:

| | |
|---|---|
| Forward C-primer: | TTCTTTGCAGGGCACCCA |
| Forward T-primer: | ATTTTTCTTTGCAGGGCACCT |
| Reverse Primer: | ATCCAGTGATGTCATAAGGCAGG |
| T-probe: | 6FAM-CCACGCAGTTAAGAGC-MGB-NFQ |
| | (fluorogenic TaqMan probe) |
| C-probe: | VIC-CACGCAGTCAAGAGC-MGB-NFQ |
| | (fluorogenic TaqMan probe) |

(6FAM—6-carboxyfluorescein; VIC-; MGB—minor groove binder; NFQ—non-fluorescent quencher.

The probes and primers used in the assay were custom synthesized by Applied Biosystems.

Genotype Calls for DNA Assay

| | |
|---|---|
| Reaction 2 (C primer) | Reaction 3 (T primer) |

| C probe | T probe | C probe | T probe | genotype |
|---|---|---|---|---|
| + | | | | CC/CC |
| + | + | | | CC/CT |
| + | | + | | CC/TC |
| + | | | + | CC/TT |
| | + | | | CT/CT |
| | + | + | | CT/TC |
| | + | | + | CT/TT |
| | | + | | TC/TC |
| | | + | + | TC/TT |
| | | | + | TT/TT |

Assay fails if the observation is not listed above

### EXAMPLE 3

### Allele Detection for RNA Using TaqMan® Minor Groove Binding Assay

For each DNA sample to be tested, two reaction mixtures are prepared by combining appropriate amount of reagents shown in [2] and [3]. The reaction mixtures are placed into separate wells of a 384-well plate and the plate is processed by the ABI Prism® 7900HT sequence detection system (Applied Biosystems) The thermocycler parameters are set as indicated by the manufacturer's instructions. When the thermocycler is done, data collected are analyzed using the Sequence Detection System (SDS) software (Applied Biosystems) to determine if the reaction is positive or negative for a particular base substitution. The genotype is then assigned to the tested sample based on either a positive or negative reaction. Detection of fluorescence from 6FAM indicates the presence of T at the SNP; fluorescence from VIC indicates the presence of C at the SNP. The genotype is then assigned to the tested sample based on either a positive or negative reaction, according to the table of genotype calls below. In the genotype column, the first pair of letters refer to the SNP at nucleotide 195 and 231, respectively, of one strand of DNA, and the second pair of letters refer to the SNP at nucleotides 195 and 231, respectively, in the other strand of DNA. A+ indicates a positive reaction. A negative reaction is indicated by a blank.

[4] PCR Reaction Mixture

| | | |
|---|---|---|
| Forward primer | 0.5 | μM |
| Reverse A primer | 0.1 | μM |
| Probe: C and T | 0.1 | μM |
| RT-PCR master mix | 10 | μl |
| Reverse transcriptase | 0.2 | μl |
| Total RNA | 100 | ng |
| H₂0 q.s. | 20 | μl |

The RT-PCR master mix is TaqMan® OneStep PCR Master Mix Kit (#4309169); Applied Biosciences)

[5] PCR Reaction Mixture

| | | |
|---|---|---|
| Forward primer | 0.5 | μM |
| Reverse G-primer | 0.1 | μM |
| Probe: C and T | 0.1 | μM |
| RT-PCR master mix | 10 | μl |
| Reverse transcriptase | 0.2 | μl |
| Total RNA | 100 | ng |
| H₂0 q.s. | 20 | μl |

(The RT-PCR Master Mix is TaqMan® OneStep PCR Master Mix Kit (#4309169); Applied Biosciences)

The thermocycler settings are as follows: 50 C° 20 min, 1 cycle; 95 C° 15 min, 1 cycle; 90° C. 15 sec; 60° C. 60 sec, 25 cycles.

The following probes and primers are used in the assay:

| | |
|---|---|
| Forward primer: | TGC CGT GGT GGG AAG CT |
| Reverse G-primer: | TCT CAG ATT TCC AGG GCT CTT G |
| Reverse A-primer: | TCT CAG ATT TCC AGG GCT CTT A |
| C-Probe: | 6FAM-ATG GGC ACC CAG CT-MGB-NFQ |
| | (fluorogenic TaqMan probe) |
| T-Probe: | VIC-ATG GGC ACC TAG CT-MGB-NFQ |
| | (fluorogenic TaqMan probe) |

The probes and primers used in the assay were custom synthesized by Applied Biosystems.

Genotype Calls for RNA Assay

| | |
|---|---|
| Reaction 4 (A primer) | Reaction 5 (G primer) |

| C probe | T probe | C probe | T probe | genotype |
|---|---|---|---|---|
| + | | | | CT/CT |
| + | + | | | CT/TT |
| + | | + | | CT/CC |
| + | | | + | CT/TC |
| | + | | | TT/TT |
| | + | + | | TT/CC |
| | + | | + | TT/TC |
| | | + | | CC/CC |
| | | + | + | CC/TC |
| | | | + | TC/TC |

Assay fails if the observation is not listed above

We claim:

1. A method of screening chickens to determine those more likely to have a lean or fat phenotype comprising the steps of
obtaining a sample of genetic material from a chicken; and
identifying the presence of at least one polymorphism in said genetic material in the thyroid hormone repressible gene or its 3′ untranslated region as shown in SEQ ID NO: 1 that is associated with a fat phenotype or a lean phenotype.

2. The method of claim 1 wherein said at least one polymorphism comprises the presence of T at the position corresponding to nucleotide 195 relative to the first nucleotide of the start codon of the THRG protein as shown in SEQ ID NO: 1 and the presence of C at the position corresponding to nucleotide 231 relative to the first nucleotide of the start codon of the THRG protein as shown in SEQ ID NO: 1 and is associated with a lean phenotype, or said at least one polymorphism comprises the presence of C at the position corresponding to nucleotide 195 relative to the first nucleotide of the start codon of the THRG protein as shown in SEQ ID NO: 1 and the presence of T at the position corresponding to nucleotide 231 relative to the first nucleotide of the start codon of the THRG protein as shown in SEQ ID NO: 1 and is associated with a fat phenotype.

3. The method of claim 1 wherein said step of identifying the presence of said polymorphism comprises the steps of
amplifying a portion of said genetic material with a forward primer and a reverse primer capable of amplifying a region of the thyroid hormone repressible gene or 3′ untranslated region as shown in SEQ ID NO: 1, which region contains a polymorphic site, and
detecting the polymorphism in said amplified region.

4. The method of claim 3 wherein said forward and reverse primers are selected from and based upon SEQ ID NO: 1 or its complementary sequence.

5. The method of claim 4 wherein said forward and reverse primers are selected from the group consisting of

| | |
|---|---|
| TTCTTTGCAGGGCACCCA; | (SEQ ID NO: 4 |
| ATTTTTCTTTGCAGGGCACCT; | (SEQ ID NO: 5) |
| ATCCAGTGATGTCATAAGGCAGG; | (SEQ ID NO: 6) |
| 6FAM-CCACGCAGTTAAGAGC- | (SEQ ID NO: 7) |
| CACGCAGTCAAGAGC | (SEQ ID NO: 8) |
| TGCCGTGGTGGGAAGCT; | (SEQ ID NO: 9) |
| TCTCAGATTTCCAGGGCTCTTG; | (SEQ ID NO: 10) |
| TCTCAGATTTCCAGGGCTCTTA; | (SEQ ID NO: 11) |
| ATGGGCACCCAGCT; | (SEQ ID NO: 12) |
| ATGGGCACCTAGCT | (SEQ ID NO: 13) |
| GTGGTGGGAAGCTGAAAT GC; | (SEQ ID NO: 14) |
| TGATGTCATAAGGCAGGAGACATC; | (SEQ ID NO: 15) |
| TCCTAAATCTGAGACCTCACTGACCACGCA. | (SEQ ID NO: 16) |

6. The method of claim 4 wherein the step of detecting comprises the step of detecting binding of a nucleotide probe to said amplified region.

7. The method of claim 6 wherein said nucleotide probe is selected from the group consisting of CCACGCAGTRAAGAGC and CACGCAGTCAAGAGC.

8. The method of claim 6 wherein said nucleotide probe further comprises a fluorophore and a quencher.

9. A method of screening chickens to identify a polymorphism associated with a fat or lean phenotype comprising
obtaining a sample of genetic material from a chicken; and
identifying the presence of at least one polymorphism in said genetic material in the thyroid hormone repressible gene-or its 3′ untranslated region as shown in SEQ ID NO: 1 that is associated with a fat phenotype or a lean phenotype.

10. The method of claim 9 wherein said at least one polymorphism comprises the presence of T at the position corresponding to nucleotide 195 relative to the first nucleotide of the start codon of the THRG protein as shown in SEQ ID NO: 1 and the presence of C at the position corresponding to nucleotide 231 relative to the first nucleotide of the start codon of the THRG protein as shown in SEQ ID NO: 1 and is associated with a lean phenotype, or said at least one polymorphism comprises the presence of C at the position corresponding to nucleotide 195 relative to the first nucleotide of the start codon of the THRG protein as shown in SEQ ID NO: 1 and the presence of T at the position corresponding to nucleotide 231 relative to the first nucleotide of the start codon of the THRG protein as shown in SEQ ID NO: 1 and is associated with a fat phenotype.

11. The method of claim 9 wherein said step of identifying the presence of said polymorphism comprises the steps of
amplifying a portion of said genetic material with a forward primer and a reverse primer capable of amplifying a region of the thyroid hormone repressible gene or 3′ untranslated region as shown in SEQ ID NO: 1, which region contains a polymorphic site, and
detecting the polymorphism in said amplified region.

12. The method of claim 11 wherein said forward and reverse primers are selected from and based upon SEQ ID NO: 1 or its complementary sequence.

13. The method of claim 12 wherein said forward and reverse primers are selected from the group consisting of

| | |
|---|---|
| TTCTTTGCAGGGCACCCA; | (SEQ ID NO: 4 |
| ATTTTTCTTTGCAGGGCACCT; | (SEQ ID NO: 5) |
| ATCCAGTGATGTCATAAGGCAGG; | (SEQ ID NO: 6) |
| 6FAM-CCACGCAGTTAAGAGC- | (SEQ ID NO: 7) |
| CACGCAGTCAAGAGC | (SEQ ID NO: 8) |
| TGCCGTGGTGGGAAGCT; | (SEQ ID NO: 9) |
| TCTCAGATTTCCAGGGCTCTTG; | (SEQ ID NO: 10) |
| TCTCAGATTTCCAGGGCTCTTA; | (SEQ ID NO: 11) |
| ATGGGCACCCAGCT; | (SEQ ID NO: 12) |
| ATGGGCACCTAGCT | (SEQ ID NO: 13) |
| GTGGTGGGAAGCTGAAAT GC; | (SEQ ID NO: 14) |
| TGATGTCATAAGGCAGGAGACATC; | (SEQ ID NO: 15) |
| TCCTAAATCTGAGACCTCACTGACCACGCA. | (SEQ ID NO: 16) |

14. The method of claim 11 wherein the step of detecting comprises the step of detecting binding of a nucleotide probe to said amplified region.

15. The method of claim 14 wherein said nucleotide probe is selected from the group consisting of CCACGCAGTTAAGAGC and CACGCAGTCAAGAGC.

16. The method of claim 14 wherein said nucleotide probe further comprises a fluorophore and a quencher.

17. An isolated oligonucleotide comprising from about 10 to about 30 contiguous bases of SEQ ID NO: 1 or SEQ ID NO: 3, or the complementary sequence of SEQ ID NO: 1 or SEQ ID NO: 3.

18. An isolated oligonucleotide sequence of claim 17 selected from the group consisting of

| | |
|---|---|
| TTCTTTGCAGGGCACCCA; | (SEQ ID NO: 4 |
| ATTTTTCTTTGCAGGGCACCT; | (SEQ ID NO: 5) |
| ATCCAGTGATGTCATAAGGCAGG; | (SEQ ID NO: 6) |
| 6FAM-CCACGCAGTTAAGAGC- | (SEQ ID NO: 7) |
| CACGCAGTCAAGAGC | (SEQ ID NO: 8) |
| TGCCGTGGTGGGAAGCT; | (SEQ ID NO: 9) |
| TCTCAGATTTCCAGGGCTCTTG; | (SEQ ID NO: 10) |
| TCTCAGATTTCCAGGGCTCTTA; | (SEQ ID NO: 11) |
| ATGGGCACCCAGCT; | (SEQ ID NO: 12) |
| ATGGGCACCTAGCT | (SEQ ID NO: 13) |
| GTGGTGGGAAGCTGAAAT GC; | (SEQ ID NO: 14) |
| TGATGTCATAAGGCAGGAGACATC; | (SEQ ID NO: 15) |
| TCCTAAATCTGAGACCTCACTGACCACGCA. | (SEQ ID NO: 16) |

19. The isolated oligonucleotide sequence of claim 17 wherein said isolated nucleotide sequence is TCCTAAATCTGAGACCTCACTGACCACGCA.

20. The isolated oligonucleotide sequence of claim 18 further comprising a fluorophore and a quencher.

21. A kit comprising at least one allele-specific oligonucleotide or gene expression product indicator.

22. An isolated polynucleotide comprising at least the coding portion of SEQ ID NO: 1.

23. The isolated polynucleotide of claim 21 wherein said polynucleotide comprises SEQ ID NO: 1

25. An isolated polypeptide comprising SEQ ID NO: 2.

25. An isolated polynucleotide comprising SEQ ID NO: 4.
